(19) United States
(12) Patent Application Publication
(10) Pub. No.: US 2012/0289866 A1
(43) Pub. Date: Nov. 15, 2012

(54) DEVICE FOR COLLECTION OF GAIT ANALYSIS DATA FOR UPPER AND LOWER EXTREMITIES

(75) Inventors: Steven E. Irby, Houston, TX (US); Z. Maria Oden, Houston, TX (US); Matthew K. Jones, Houston, TX (US); Marcia K. O'Malley, Houston, TX (US); Michele Lynn Pyle, Albuquerque, NM (US); Andrew S. Berger, Houston, TX (US); Judith Lynn Linton, Houston, TX (US)

(73) Assignees: Shriners Hospital for Children, Tampa, FL (US); William Marsh Rice University, Houston, TX (US)

(21) Appl. No.: 13/446,655

(22) Filed: Apr. 13, 2012

Related U.S. Application Data
(60) Provisional application No. 61/475,096, filed on Apr. 13, 2011.

(51) Int. Cl. A61B 5/11

(52) U.S. Cl. 600/595

(57) ABSTRACT

The present disclosure relates to a system for collecting gait analysis data for analysis. The system may utilize force plate or balance boards as well as strain gauge force measurements to improve physical therapy and gait analysis for patients that use physical supports to walk or stand. The device measures force applied to the floor and to handrails to provide data on the patient's use of aid, e.g., the handrail, while standing or walking For physical therapy implementations, the system may also be combined with a game or other visual interface that provides feedback to the patient and/or to the caregiver.

### CROSS-REFERENCE TO RELATED APPLICATIONS

This application claims the benefit of U.S. Provisional Patent Application No. 61/475,096, entitled “DEVICE FOR COLLECTION OF GAIT ANALYSIS DATA FOR BOTH UPPER AND LOWER BODY EXTREMITIES,” and filed Apr. 13, 2011, which is herein incorporated by reference in its entirety for all purposes.

### BACKGROUND

The present disclosure relates generally to the quantification of dexterity in people with motor impairment.

It may be difficult to acquire data related to the use of or reliance on walking aids by a patient. For example, commercially available balance therapy systems are not known which collect information that would indicate a patient's need for, or reliance on, an aid for balance or walking

### SUMMARY

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

The present disclosure relates to a system for collecting gait analysis data for analysis. The system may utilize force plate or balance boards as well as strain gauge force measurements to improve physical therapy and gait analysis for patients that use physical supports to walk or stand. The device measures force applied to the floor and to handrails to provide data on the patient's use of aid, e.g., the handrail, while standing or walking For physical therapy implementations, the system may also be combined with a game or other visual interface that provides feedback to the patient and/or to the caregiver.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 depicts a gait analysis system, in accordance with aspects of the present disclosure.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Further, the calculations, correlations, and/or measurements discussed herein may be performed using one or more suitable computer-implemented algorithms, such as may be stored on a computer-readable memory or medium for execution by a suitable processing component in communication with the medium or memory. In addition, previously determined constants or correlations may be stored on the computer-readable memory or medium and accessed by the processing component during execution of the algorithms. The processor may also receive inputs or measurements from a measurement device, such as a spectrophotometer, or from personnel. Such inputs may be processed used as inputs to the algorithms during execution of the algorithms. Examples of devices having suitable processing components, memory components, user interface circuitry, and/or circuitry for accessing local or remote media include, but are not limited to, desktop, notebook, and tablet computers, personal digital assistants, cellular telephones, media players, and so forth.

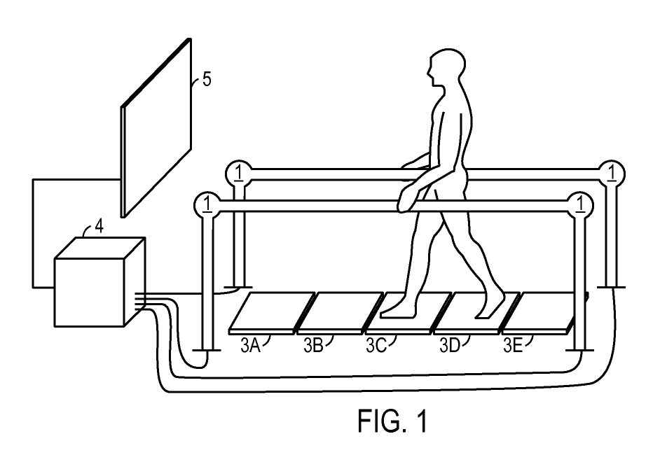

Turning to FIG. 1, in one embodiment, a device is disclosed that combines balance board 3 (such as a Nintendo Wii® balance board) or force plate technology and strain gauge force measurement to improve physical therapy and gait analysis for patients who require physical supports to walk or stand. The device measures the forces applied to the floor and handrails of the device and can compare these measurements to each other or to previous test results. These comparisons provide visibility to a patient's progress and can increase the overall effectiveness of physical therapies.

Track Layout: The walking track, in one implementation, uses a combination of measurement from the strain gauge force measurement technology associated with a balance board 3 and other bending beam strain gauges. For example, in one implementation, five Wii® balance boards 3 may be set into the floor track housing beneath durable, sectioned, and color-coded plastic paneling. In one implementation, the balance boards 3 are set on tracks to allow free motion in the x- and y-directions (z-direction is perpendicular to the horizontal plane). Beneath each board 3 is a horizontal force measurement beam at each edge of the board 3. In another implementation, feet instrumented with gauges may be placed underneath the balance boards 3 to measure shear forces. In certain of these embodiments, the boards 3 are stationary.

Track Casing: The walking track may be housed in a plywood or eucaboard rectangular box. In one implementation, any flat, relatively lightweight board (such as board about ¼″ in thickness) could be used. In one embodiment, the housing for three National Instruments® 9219 DAQ cards is located at one end of the track casing. Each of the four channels on the 9219 DAQ cards collects data from each of the four handles in the x-, y-, or z-direction. The DAQ cards are connected to a PC 4 running a stand-alone Virtual Instrument, created using National Instruments LabVIEW 2009. This stand alone Virtual Instrument collects and appropriately manipulates signals to provide the overall force applied to each handrail in each of the x-, y-, and z-directions. The connection/supports for the handrails are affixed on the outside vertical +x and −x walls of the casing. In other contemplated approaches, a load cell circuit with its own power supply, PC connection, and filter may be employed in place of an all-purpose DAQ card.

Handrails: In one implementation, the device includes a set of two handrails, which run lengthwise along the balance board walkway. The handrails, in one embodiment, have an outer diameter of 1.25″ and may be constructed from materials used in hospital grade patient handrails. The sensors 1 for handrail forces are located at either end of the walkway on both handrails. Handrail sensors 1 may be composed of a pinwheel orientation of load cells, instrumented to measure in the x-, y-, and z-directions, and affixed inside the sensor housing.

Monitors and Video Interface: A monitor 5 for the device is used to show the display from a desktop PC 4, or other connected computer or electronic device. Additional monitors 5 can be added to simultaneously display the diagnostic interface for doctors and therapists and any video game display for the patient. The balance boards 3 collect data on the user's center of balance as they move along the track. This progress is plotted on the diagnostic interface, so that the patient can see on the screen a map depicting where his or her center of balance is on the walkway. In one embodiment, a circle representing each of the two handrails will be located next to both color and numeric indicators; these indicators will display the magnitude of force the patient is applying to each rail in each of the x-, y-, and z-directions. In other embodiments, indicators (circular or otherwise) may be provide, one of which indicates positive forces, another of which indicates negative forces.

The present device is intended to be used in a gait analysis lab and/or physical therapy lab. The device will provide engaging and entertaining physical therapy game play sessions for a suitable time period, such as for twenty minutes or more. Such a session may involve asking the patient to balance in place, walk the track, and walk the track and turn around. Game play may encourage a patient to work towards walking or standing on his own by subtracting from game success when the handrail supports are used.

The device can also serve as a controller for a gaming system or environment. In particular, the present device incorporate handrails with balance based gaming. This element of handrails adds both new possibilities for games and the opportunity for unimpaired children to participate in a part of a sibling's or friend's recovery process.

One feature of the present disclosure is the use of instrumented walking aids to collect data on a patient's use of aid. Other instrumented aid devices, such as instrumented stair handrails have not been incorporated in commercially available balance therapy systems to collect this kind of data. This device will be able to show doctors, therapists and patients how and how-much the patient relies on walking aids, which will allow better administration of physical therapy techniques. The data will help determine which activities and motions the patient has trouble with so that physical therapy can be further customized.

Involving video games in physical therapy techniques may be useful for increasing patient's interest in the therapy. The special challenges for patients who cannot stand, walk, or turn about on their own have previously been a barrier to video game balance therapy. The present device may overcome this barrier by providing both support integrated with the gaming system, so that balance challenged patients can use the device safely, as well as quantifying data on the patient's progress toward independent balance. In addition, this device uses signals routed through a PC 4, so that new games can be easily created for it.

In one specific implementation, the device is made by first constructing a wooden housing for the balance board walkway from plywood, wood glue, and nails. The device may also include walkway ramps, so that patients who cannot make the step up onto the balance boards 3 can use the device. The balance boards 3 are then placed inside the walkway, along with rectangular plexiglass coverings to bridge the gaps between the boards 3. Foam filler may, if needed, be placed between the plexiglass sheets to fill in the gaps between them without transmitting forces to the adjacent board 3. Each board 3 is loaded with a plug in battery pack, as opposed to double A batteries, so that the device does not have to be disassembled when batteries die. The bottom of the housing may, in some implementations, include access ports, so that the user may reach the Bluetooth “sync” button without disassembling the walkway. The signal from the balance boards 3 is connected to a PC 4 running the diagnostic interface using a seven signal Bluetooth USB adapter. This adapter collects each signal as a separate input.

In this implementation the vertical bars of the handrail structure are attached to the walkway at either end of the walkway on each side, where they slide into a casing. This casing includes round pipe sections where the vertical handrail bars are placed, as well as reinforcement running underneath the walkway to the other handrail supports. The handrail force sensors 1 are attached to the top of the vertical handrail bars. The sensors 1 include four load cells, as well as a round holder for the horizontal bar of the structure (to which the patient holds on during use). This holder maintains appropriate contact with the load cells so that the handrails may be assembled and disassembled without having to reassemble the handrail sensor box. The signals from the handrail sensors 1 are wired to the 9219 DAQ cards, sitting next to the walkway. These cards are connected via USB to the PC 4 running the diagnostic interface.

In certain of the embodiments discussed herein, the balance boards 3 used may provide less accurate measurement than more expensive force plates. The balance boards 3, however, may provide a sufficiently accurate representation of a user's center of balance to provide the desired assessment and feedback. One way to improve accuracy of the system, if desired, may be to use the handrail sensor portion of the system in conjunction with a force plate for robust balance testing. However, to the extent that the device is used to provide a way for patients to practice independent balance while discouraging reliance on walking aids, this modification may not be needed as any limitation in accuracy should not hinder the effectiveness of the device.

As will be appreciated, the preceding describes certain specific embodiments to illustrate approaches that may be employed. However, the above discussion is not intended to limit the design of the contemplated device and variations to the design could include alterations to either the walkway or handrail sensors. Similarly, different kinds of load cell schemes could be developed for the walkway center-of-balance measurement. Variations to the handrails could include any number of different load cell designs measuring forces in three directions, as well as a stand-alone handrail measurement system to be used with a more accurate force plate measurement system or an infrared camera motion detection system.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art, including combinations of aspects or features of the embodiments and examples disclosed herein. Such other examples are intended to be within the scope of the claims if they have structural elements that do not differ from the literal language of the claims, or if they include equivalent structural elements with insubstantial differences from the literal languages of the claims. The specific embodiments described above have been shown by way of example, and it should be understood that these embodiments may be susceptible to various modifications and alternative forms, including combinations of various features and aspects of the examples or embodiments discussed herein. It should be further understood that the claims are not intended to be limited to the particular forms disclosed, but rather to cover all modifications, equivalents, and alternatives falling within the spirit and scope of this disclosure.

1. A method for assessing patient use of support, comprising the acts of:
acquiring a first set of data from one or more balance board or force plates on which a patient stands or walks;
acquiring a second set of data from one or more handrails positioned such that the patient can reach the one or more handrails when the patient is on the one or more balance board or force plates; and
assessing the patients reliance on the one or more handrails for standing or moving using the first set of data and the second set of data.

2. The method of claim 1, comprising providing physical therapy to the patient while acquiring the first set of data and the second set of data.

3. The method of claim 1, comprising displaying a game or other visual feedback to the patient while acquiring the first set of data and the second set of data.

4. The method of claim 1, comprising displaying a center of balance of the patient.

5. The method of claim 1, comprising displaying the magnitude and direction of force applied to each handrail by the patient.

6. A gait analysis system, comprising:
one or more balance boards or force plates disposed within a track housing, wherein the one or more balance boards or force plates generate force and position data in response to a person standing or moving on the one or more balance boards or force plates; and
one or more handrails provided within reach of the one or more balance boards or force plates such that the person standing or moving on the one or more balance boards or force plates can reach the one or more handrails, wherein the one or more handrails generate force and position data in response to contact by the person.

7. The gait analysis system of claim 6, comprising a monitor configured to display a game or other visual feedback to the person.

8. The gait analysis system of claim 7, wherein the game or other visual feedback includes an indication of the patient's center of balance.

9. The gait analysis system of claim 7, wherein the game or other visual feedback includes an indication of the magnitude of force applied by the patient to each handrail in each of the x-, y-, and z-directions.

10. The gait analysis system of claim 6, wherein the balance boards or force plates move freely in the x-dimension and y-dimension.

11. The gait analysis system of claim 6, wherein the handrails move freely in the x-dimension, y-dimension, and z-dimension.

12. The gait analysis system of claim 6, comprising a horizontal force measurement beam at each edge of the one or more balance boards or force plates.

13. The gait analysis system of claim 6, comprising one or more multi-channel data acquisition cards configured to collect the force and position data from one or both of the one or more handrails or the one or more balance boards or force plates.

14. The gait analysis system of claim 13, comprising a computer configured to collect data from the one or more data acquisition cards and to process the collected data.

15. The gait analysis system of claim 6, wherein the one or more handrails are composed of a pinwheel orientation of load cells instrumented to measure in the x-, y-, and z-directions.

16. A system, comprising:
one or more floor segments that generate a two-dimensional measure of force representing the position or movement of a patient over time with respect to the one or more segments;
one or more handrails that generate a three-dimensional measure of force representing contact between the patient and the one or more handrails over time; and
a monitor configured to provide visual cues to the patient while the patient is on the one or more floor segments.

17. The system of claim 16, comprising a computer configured to acquire and process the two-dimensional measures and the three-dimensional measures.

18. The system of claim 16, wherein the visual cues comprise a game in which walking or standing without contacting the one or more handrails is rewarded.

19. The system of claim 16, wherein the visual cues comprise a game in which contacting the one or more handrails is discouraged.

20. The system of claim 16, wherein the visual cues comprise one or more of an indication of a center of balance of the patient or a magnitude and direction of force applied to the one or more handrails.
